(19) United States
(12) Patent Application Publication
(10) Pub. No.: US 2006/0064071 A1
(43) Pub. Date: Mar. 23, 2006

(54) Gas inflation/evacuation system incorporating a reservoir and removably attached sealing system for a guidewire assembly having an occlusive device

(75) Inventors: Michael J. Bonnette, Minneapolis, MN (US); Eric J. Thor, Arden Hills, MN (US); Richard R. Prather, St. Michael, MN (US); Robert G. Dutcher, Maple Grove, MN (US)

Correspondence Address: HUGH D JAEGER, 1000 SUPERIOR BLVD, SUITE 302, WAYZATA, MN, 553911873, US

(73) Assignee: Possis Medical, Inc.

(21) Appl. No.: 11/009,810

(22) Filed: Dec. 10, 2004

Related U.S. Application Data
(63) Continuation-in-part of application No. 10/838,464, filed on May 4, 2004.
Application No. 10/838,464 is a continuation-in-part of application No. 10/012,903, filed on Nov. 6, 2001, now Pat. No. 6,932,828.
Application No. 10/838,464 is a continuation-in-part of application No. 10/012,891, filed on Nov. 6, 2001.
Application No. 10/838,464 is a continuation-in-part of application No. 10/007,788, filed on Nov. 6, 2001, now Pat. No. 6,942,678.

(51) Int. Cl. A61M 31/00

(52) U.S. Cl. 604/500; 604/97.01

(57) ABSTRACT

A gas inflation/evacuation system incorporating a reservoir and removably attached sealing system for a guidewire assembly having an occlusive device and method. A gas inflation/evacuation system is removably connectible to a proximal portion of a guidewire assembly where a sealing system interfaces and cooperatively interacts between the gas inflation/evacuation system and the proximal portion of the guidewire assembly to provide for repeated inflation and deflation of an occlusive balloon to provide a hubless guidewire assembly over which ablation and other type catheters can be loaded.

### CROSS REFERENCES TO RELATED APPLICATIONS

This application is a continuation-in-part of U.S. patent application Ser. No. 10/838,464, filed Apr. 29, 2004, entitled “Gas Inflation/Evacuation System and Sealing System for Guidewire Assembly Having Occlusive Device,” which is a continuation-in-part of U.S. patent application Ser. No. 10/012,903, filed Nov. 6, 2001, entitled “Guidewire Occlusion System Utilizing Repeatably Inflatable Gas-Filled Occlusive Device,” and U.S. patent application Ser. No. 10/012,891, filed Nov. 6, 2001, entitled “Guidewire Assembly Having Occlusive Device and Repeatably Crimpable Proximal End,” and U.S. patent application Ser. No. 10/007,788, filed Nov. 6, 2001, entitled “Gas Inflation/Evacuation System and Sealing System for Guidewire Assembly Having Occlusive Device,” all of which are hereby incorporated herein by reference.

### BACKGROUND OF THE INVENTION

1. Field of the Invention

The present invention relates generally to the field of vascular medical devices. More specifically, the present invention relates to a gas inflation/evacuation system incorporating a reservoir and a removably attached sealing system incorporating a compression sealing mechanism and an inflation tube sealing/crimping mechanism for selectively and repeatedly inflating an occlusive balloon and for sealing and crimping an extended guidewire sealable section at the proximal end of a guidewire during an occlusive procedure.

2. Description of the Prior Art

Arterial disease involves damage that happens to the arteries in the body. Diseased arteries can become plugged with thrombus, plaque, or grumous material that may ultimately lead to a condition known as ischemia. Ischemia refers to a substantial reduction or loss of blood flow to the heart muscle or any other tissue that is being supplied by the artery and can lead to permanent damage of the affected region. While arterial disease is most commonly associated with the formation of hard plaque and coronary artery disease in the heart, similar damage can happen to many other vessels in the body, such as the peripheral vessels, cerebral vessels, due to the buildup of hard plaque or softer thrombus or grumous material within the lumen of an artery or vein.

A variety of vascular medical devices and procedures have been developed to treat diseased vessels. The current standard procedures include bypass surgery (where a new blood vessel is grafted around a narrowed or blocked artery) and several different types of non-surgical interventional vascular medical procedures, including angioplasty (where a balloon on a catheter is inflated inside a narrowed or blocked portion of an artery in an attempt to push back plaque or thrombotic material), stenting (where a metal mesh tube is expanded against a narrowed or blocked portion of an artery to hold back plaque or thrombotic material), and debulking techniques in the form of atherectomy (where some type of high speed or high power mechanism is used to dislodge hardened plaque) or thrombectomy (where some type of mechanism or infused fluid is used to dislodge grumous or thrombotic material). In each of these interventional vascular medical procedures, a very flexible guidewire is routed through the patient's vascular system to a desired treatment location and then a catheter that includes a device on the distal end appropriate for the given procedure is tracked along the guidewire to the treatment location.

Although interventional vascular procedures avoid many of the complications involved in surgery, there is a possibility of complications if some of the plaque, thrombus or other material breaks free and flows downstream in the artery or other vessel, potentially causing a stroke, a myocardial infarction (heart attack), or other tissue death. One solution to this potential complication is to use some kind of occlusive device to block or screen the blood flowing downstream of the treatment location. Examples of catheter arrangements that use a pair of balloons as occlusive devices to create an isolated space in the blood vessel are described in U.S. Pat. Nos. 4,573,966, 4,636,195, 5,059,178, 5,320,604, 5,833,644, 5,925,016, 6,022,336 and 6,176,844. Examples of catheter arrangements that use a single balloon as an occlusive device either upstream or downstream of the treatment location are described in U.S. Pat. Nos. 5,171,221, 5,195,955, 5,135,482, 5,380,284, 5,688,234, 5,713,917, 5,775,327, 5,792,179, 5,807,330, 5,833,650, 5,843,022, 6,021,340, 6,159,195 and 6,248,121. An example of a catheter arrangement that uses a mechanically-expanded occlusive device is shown in U.S. Pat. No. 6,231,588. Occlusive balloons also have been used on non-over-the-wire catheters without any guidewire internal to the catheter as described, for example, in U.S. Pat. Nos. 4,838,268 and 5,209,727.

The use of an occlusive device as part of a vascular procedure is becoming more common in debulking procedures performed on heart bypass vessels. Most heart bypass vessels are harvested and transplanted from the saphenous vein located along the inside of the patient's leg. The saphenous vein is a long, straight vein that has a capacity more than adequate to support the blood flow needs of the heart. Once transplanted, the saphenous vein is subject to a buildup of plaque or thrombotic materials in the grafted arterial lumen. Unfortunately, the standard interventional vascular treatments for debulking are only moderately successful when employed to treat saphenous vein coronary bypass grafts. The complication rate for a standard balloon angioplasty procedure in a saphenous vein coronary bypass graft is higher than in a native vessel with the complications including embolization, “no-reflow” phenomena, and procedural related myocardial infarction. Atherectomy methods including directional, rotational, and laser devices are also associated with a high degree of embolization resulting in a greater likelihood of infarction. The use of stents for saphenous vein coronary bypass grafts has produced mixed results. Stents provide for less restenosis, but they do not eliminate the risk of embolization and infarction incurred by standard balloon angioplasty.

In order to overcome the shortcomings of these standard non-surgical interventional treatments in treating saphenous vein coronary bypass graft occlusion, embolic protection methods utilizing a protective device distal to the lesion have been developed. The protective device is typically a filter or a balloon. Use of a protective device in conjunction with an atherectomy or thrombectomy device is intended to prevent emboli from migrating beyond the protective device and to allow the embolic particles to be removed, thereby subsequently reducing the risk of myocardial infarction. When the occlusive device is a balloon, the balloon is inserted and inflated at a point distal to the treatment site or lesion site. Therapy is then performed at the treatment site and the balloon acts to block all blood flow which prevents emboli from traveling beyond the balloon. Following treatment, some form of particle removal device must be used to remove the dislodged emboli prior to balloon deflation. U.S. Pat. No. 5,843,022 uses a balloon to occlude the vessel distal to a lesion or blockage site. The occlusion is treated with a high pressure water jet, and the fluid and entrained emboli are subsequently removed via an extraction tube. U.S. Pat. No. 6,135,991 describes the use of a balloon to occlude the vessel allowing blood flow and pressure to prevent the migration of emboli proximally from the treatment device.

There are various designs that have included an occlusive balloon on the end of a guidewire. U.S. Pat. Nos. 5,520,645, 5,779,688 and 5,908,405 describe guidewires having removable occlusive balloons on a distal end. U.S. Pat. No. 4,573,470 describes a guidewire having an occlusive balloon where the guidewire is bonded inside the catheter as an integral unit. U.S. Pat. Nos. 5,059,176, 5,167,239, 5,520,645, 5,779,688 and 6,050,972 describe various guidewires with balloons at the distal end in which a valve arrangement is used to inflate and/or deflate the balloon. U.S. Pat. No. 5,908,405 describes an arrangement with a removable balloon member that can be repeatedly inserted into and withdrawn from a guidewire. U.S. Pat. No. 5,776,100 describes a guidewire with an occlusive balloon adhesively bonded to the distal end with an adapter on the proximal end to provide inflation fluid for the occlusive balloon.

Except in the case of the normal cerebral anatomy where there are redundant arteries supplying blood to the same tissue, one of the problems with using an occlusive device in the arteries is that tissue downstream of the occlusive device can be damaged due to the lack of blood flow. Consequently, an occlusive device that completely blocks the artery can only be deployed for a relatively short period of time. To overcome this disadvantage, most of the recent development in relation to occlusive devices has focused on devices that screen the blood through a filter arrangement. U.S. Pat. Nos. 5,827,324, 5,938,672, 5,997,558, 6,080,170, 6,171,328, 6,203,561 and 6,245,089 describe various examples of filter arrangements that are to be deployed on the distal end of a catheter system. While a filter arrangement is theoretically a better solution than an occlusive device, in practice such filter arrangements often become plugged, effectively turning the filter into an occlusive device. The filter arrangements also are mechanically and operationally more complicated than an occlusive balloon device in terms of deployment and extraction.

As is the case in almost all angioplasty devices or stenting catheter devices where a balloon is used to expand the blood vessel or stent, most catheter occlusive balloons as well as most guidewire occlusive balloons utilize a liquid fluid such as saline or saline mixed with a radiopaque marker for fluoroscopic visualization (i.e., contrast) as the inflation medium. Generally, a liquid fluid medium for expanding vascular balloons has been preferred because the expansion characteristics of a liquid are more uniform and predictable, and because a liquid medium is easier to work with and more familiar to the doctors. In the case of angioplasty balloons, for example, high-pressure requirements (up to 20 atmospheres) necessitate that the inflation fluid be an incompressible fluid for safety reasons. While having numerous advantages, liquid fluids do not lend themselves to rapid deflation of an occlusive balloon because of the high resistance to movement of the liquid in a long small diameter tube. In the context of angioplasty procedures, the balloon catheter has a much larger lumen than a guidewire. Consequently, rapid deflation is possible. In the context of a guidewire, however, liquid filled occlusive balloons typically cannot be deflated in less than a minute and, depending upon the length of the guidewire, can take up to several minutes to deflate. Consequently, it is not practical to shorten the period of total blockage of a vessel by repeatedly deflating and then re-inflating a liquid filled occlusive balloon at the end of a guidewire.

Gas-filled balloons have been used for intra-aortic occlusive devices where rapid inflation and deflation of the occlusive device is required. Examples of such intra-aortic occlusive devices are shown in U.S. Pat. Nos. 4,646,719, 4,733,652, 5,865,721, 6,146,372, 6,245,008 and 6,241,706. While effective for use as an intra-aortic occlusive device, these occlusive devices are not designed for use as a guidewire as there is no ability to track a catheter over the intra-aortic occlusive device.

An early catheter balloon device that utilized a gas as an inflation medium and provided a volume limited syringe injection system is described in U.S. Pat. No. 4,865,587. More recently, a gas-filled occlusive balloon on a guidewire is described as one of the alternate embodiments in U.S. Pat. No. 6,217,567. The only suggestion for how the guidewire of the alternate embodiment is sealed is a valve type arrangement similar to the valve arrangement used in a liquid fluid embodiment. A similar gas-filled occlusive balloon has been described with respect to the Aegis Vortex™ system developed by Kensey Nash Corporation. In both U.S. Pat. No. 6,217,567 and the Aegis Vortex™ system, the gas-filled occlusive balloon is used for distal protection to minimize the risk of embolization while treating a blocked saphenous vein coronary bypass graft. Once deployed, the occlusive balloon retains emboli dislodged by the atherectomy treatment process until such time as the emboli can be aspirated from the vessel. No specific apparatus are shown or described for how the gas is to be introduced into the device or how the occlusive balloon is deflated.

Although the use of occlusive devices has become more common for distal embolization protection in vascular procedures, particularly for treating a blocked saphenous vein coronary bypass graft, all of the existing approaches have significant drawbacks that can limit their effectiveness. Liquid filled occlusive balloons can remain in place too long and take too long to deflate, increasing the risk of damages downstream of the occlusion. Occlusive filters are designed to address this problem, but suffer from blockage problems and can be complicated to deploy and retrieve and may allow small embolic particles to migrate downstream. Existing gas-filled occlusive balloons solve some of the problems of liquid filled occlusive balloons, but typically have utilized complicated valve and connection arrangements. It would be desirable to provide for an occlusive device that was effective, simple, quick to deploy and deflate, and that could overcome the limitations of the existing approaches.

Some of these problems have been previously addressed in three commonly owned and assigned co-pending applications, which are hereby incorporated by reference herein: U.S. patent application Ser. No. 10/838,464, filed Apr. 29, 2004, entitled “Gas Inflation/Evacuation System and Sealing System for Guidewire Assembly Having Occlusive Device,” U.S. patent application Ser. No. 10/012,903, filed Nov. 6, 2001, entitled “Guidewire Occlusion System Utilizing Repeatably Inflatable Gas-Filled Occlusive Device,” U.S. patent application Ser. No. 10/012,891, filed Nov. 6, 2001, entitled “Guidewire Assembly Having Occlusive Device and Repeatably Crimpable Proximal End,” and U.S. patent application Ser. No. 10/007,788, filed Nov. 6, 2001, entitled “Gas Inflation/Evacuation System and Sealing System for Guidewire Assembly Having Occlusive Device.”

### SUMMARY OF THE INVENTION

The general purpose of the present invention is a gas inflation/evacuation system incorporating a reservoir and removably attached sealing system for a guidewire assembly having an occlusive device. An embodiment set forth herein includes a gas inflation/evacuation system removably connectible to a proximal portion of a guidewire assembly having a guidewire that defines a lumen. A sealing system interfaces and cooperatively interacts between the gas inflation/evacuation system and the proximal portion of the guidewire assembly.

According to one embodiment of the present invention, there is provided a gas inflation/evacuation system includes a first syringe arrangement having an evacuation syringe for selectively evacuating the guidewire lumen and other regions, a reservoir arrangement for supply of an abundance of biocompatable gas inflation medium from a reservoir for multiple inflations of an occlusive device, and a second syringe arrangement that includes an inflation syringe which selectively communicates with the reservoir for inflation medium supply and which selectively introduces a biocompatable gas inflation medium into the guidewire lumen to inflate an occlusive balloon in fluid communication with the guidewire lumen of the guidewire assembly where such occlusive balloon is located proximate a distal end of the guidewire assembly. Further, a compression sealing mechanism, part of the sealing system, may be removably connected to the proximal portion of the guidewire assembly including the guidewire lumen. The compression sealing mechanism serves as a sealed interface between the guidewire assembly and the gas inflation/evacuation system where such sealed interface is suitably sealed to allow inflation of the occlusive balloon.

The compression sealing mechanism seals about a proximally located extended sealable section of the guidewire assembly but can seal around multiple elongated elements that puncture a seal residing in the compression sealing mechanism so that the compression sealing mechanism can be used a plurality of times without replacing internal seals of the compression sealing mechanism and thus is superior to other sealing mechanisms that can seal around only two wires, tubes, or other elongated elements. Further, the compression sealing mechanism may be used to seal around the extended sealable section of the guidewire of the guidewire assembly that cooperates with the gas inflation/evacuation system located proximal to the seal located internally within the compression sealing mechanism. The sealing system also includes an inflation tube sealing/crimping mechanism which accommodatingly receives the proximal portion of the guidewire assembly including the extended sealable section for crimping and sealing during use of the invention. The inflation tube sealing/crimping mechanism also severs the crimped sealed section of the guidewire subsequent to desired inflation of the occlusive balloon at the distal location on the guidewire to maintain the occlusive balloon in an inflated state and to present a proximal guidewire end unencumbered by external interfering structure suitable for accommodation of a catheter, a hub or other such devices requiring the use of guidance to a vascular site requiring the removal of thrombus, lesion, plaque, or the like.

When deflation of the occlusive balloon is desired, the portion of the crimped sealed section just distal of the crimp is cut to open the lumen, thereby allowing the occlusive balloon to readily deflate. The intact proximal portion of the guidewire may subsequently be reinserted into the seal to re-engage the gas inflation/evacuation system for another inflation of the occlusive balloon, whereby the seal effectively seals around the extended sealable section of the guidewire. Each time a deflation of the occlusive device is desired in order to reestablish blood flow to the vessel downstream of the occlusive device, the extended sealable section is cut distal to the location of the last crimp so as to quickly deflate the occlusive device. The compression sealing mechanism is readily adaptable to systems as described in U.S. patent application Ser. Nos. 10/012,903, 10/012,891 and 10/007,788. As disclosed in those patent applications, a gas inflation/evacuation system is combined with a sealing system that includes a crimping mechanism and a sealing mechanism, and these systems are removably connectible to a guidewire assembly having an occlusive device located near its distal end. The gas inflation/evacuation system is removably connectible to the proximal end of the guidewire assembly and comprises an evacuation system which includes means for evacuating the guidewire assembly and an inflation system which includes means for introducing a gas under pressure into the guidewire assembly to inflate the occlusive device, such as an occlusive balloon, a plurality of times.

The resilient seal located in the compression sealing mechanism receives and seals around the proximal portion of the guidewire, and the compression sealing mechanism includes compression structure that compresses at least a portion of the resilient seal. The compressive force is transmitted into the seal and compresses the seal around tubes, wires, or other elongated elements that pass through the seal. One guidewire or a plurality of guidewires may penetrate the seal, e.g., between two and ten, or at least three. The compression sealing mechanism operates automatically; that is, when the resilient seal is penetrated by a tube, wire, or other elongated element inserted therethrough, the compressed resilient seal automatically causes sealing around the element that has penetrated it.

An example of apparatus for compressing the seal is an assembly of axially related backing or retaining members that include sealing surfaces that contact the seal. A compressive force is applied to the seal via the sealing surfaces. Examples of backing or retaining members are threaded members such as nuts, caps, screw followers, and sealing glands. Further examples of backing or retaining members for applying a compressive force could include springs, tensioned parts, and biased members. For example, a disc-shaped seal may be compressed between two planar sealing surfaces, e.g., one planar sealing surface being a sealing face on a sealing cap and the other planar sealing surface being a sealing seat in a sealing gland. A specific type of seal can be one made of a resilient material, e.g., a polymer, for example, an elastomeric polymer. Seals of various geometries are contemplated, e.g., disc-shaped, spherical, and polygonal. Examples of the thickness of a seal are from about 0.030 inch to about 0.200 inch.

An advantage of the present invention is that the occlusive device can be repeatably inflated and deflated a plurality of times during a vascular procedure in between which the proximal end of the guidewire is free of mechanical connections and obstructions and, therefore, the guidewire can function as a conventional exchange guidewire for one or more over-the-wire catheters. Alternatively, the guidewire can be shorter in length for use with rapid exchange catheter systems. Unlike operation of existing liquid filled occlusive devices, the present invention enables repeated and quick inflation and deflation which allows an operator to deploy the gas-filled occlusive device numerous times during a procedure for shorter periods of time, thereby reducing the risk of potential damage to downstream tissue. Unlike operation of other gas-filled occlusive devices, the simplicity of the present invention permits the guidewire to be used as a conventional exchange guidewire. There are no complicated mechanical arrangements or valve systems internal to the guidewire that increase the cost, complexity, and potential for failure of the system.

In a preferred embodiment of the gas inflation/evacuation system incorporating a reservoir and removably attached sealing system for a guidewire assembly having an occlusive device there is included a gas inflation/evacuation system and a sealing system which includes an inflation tube sealing/crimping mechanism. The inflation/evacuation system includes a reservoir, containing a sufficient volume of biocompatable gas for multiple inflations of an occlusive device, an inflation syringe, an evacuation syringe, an inflation valve, a vacuum valve, check valves, a pressure gauge and connective tubing and multiple connectors. For safety purposes, the inflation syringe is appropriately sized to contain just enough inflation medium to inflate the occlusive balloon so as to minimize the volume of biocompatable gas in the gas inflation/evacuation system in the event of a leak. The sealing system is comprised of an inflation tube sealing/crimping mechanism and a compression sealing mechanism; and the guidewire assembly includes a guidewire having a lumen, a flexible tip, an inflatable occlusive balloon, and an extended sealable section.

In a preferred embodiment, the extended sealable section of the guidewire is an extended crimpable section which is accommodated by and which interacts with the sealing system including the inflation tube sealing/crimping mechanism as well as the compression sealing mechanism. The extended crimpable section has a sufficient length to permit a plurality of crimps and cuts along the extended crimpable section and preferably has an outer diameter that is smaller than the outer diameter of the main body portion of the guidewire. The inflation tube sealing/crimping mechanism is used to crimp, seal and sever the extended crimpable section of the guidewire to seal the guidewire a plurality of times. Preferably, the gas inflation/evacuation system and the inflation tube sealing/crimping mechanism and the compression sealing mechanism of the sealing system constitute a handheld apparatus. Alternatively, the sealing system composed of the inflation tube sealing/crimping mechanism and the compression sealing mechanism may be a handheld unit completely separate from the gas inflation/evacuation system. Preferably, the extended crimpable section of the guidewire is dimensioned and the inflation tube sealing/crimping mechanism is arranged such that an effective outer diameter of the extended crimpable section at the location of a seal is no greater than the outer diameter of the main body portion of the guidewire when the extended crimpable section is sealed.

Several significant aspects and features of the present invention include a gas inflation/evacuation system, a sealing system, and a guidewire assembly, where the sealing system includes an inflation tube sealing/crimping mechanism and a compression sealing mechanism.

Another significant aspect and feature of the present invention is a gas inflation/evacuation system having a reservoir which cooperatively interacts with an inflation syringe for inflation of an occlusive balloon in the guidewire assembly.

Yet another significant aspect and feature of the present invention is the use of an inflation syringe which, for purposes of safety, is appropriately sized to inject no more inflation medium into a occlusive balloon than required in case of occlusive balloon leakage or breakage.

Another significant aspect and feature of the present invention is a gas inflation/evacuation system having an evacuation syringe which cooperatively interacts to evacuate a guidewire assembly.

Another significant aspect and feature of the present invention is a gas inflation/evacuation system having positionable valves and having check valves incorporated for selective supplying of inflational medium from a reservoir, for selective evacuation of connected components of the invention, and for selective pressurization of various components of the invention.

Another significant aspect and feature of the present invention is a gas inflation/evacuation system incorporating a check valve to prevent injection of air into a conduit.

Another significant aspect and feature of the present invention is a gas inflation/evacuation system incorporating check valves to minimize the number of components.

Another significant aspect and feature of the present invention is a gas inflation/evacuation system incorporating a pressure gauge to monitor inflation and evacuation procedures.

Another significant aspect and feature of the present invention is a gas inflation/evacuation system which cooperatively interacts with a compression sealing mechanism to sealingly and temporarily connect the gas inflation/evacuation system to the guidewire assembly.

Another significant aspect and feature of the present invention is the incorporation of an inflation tube sealing/crimping mechanism to cooperatively interact with the compression sealing mechanism to seal and sever an extended sealable section of a guidewire having a lumen in order to maintain inflation of an occlusive balloon at a distal location on the guidewire assembly.

Another significant aspect and feature of the present invention is the provision for repeatable inflation and deflation of an occlusive balloon multiple times.

Another significant aspect and feature of the present invention is a resilient seal with a compression sealing mechanism that automatically and effectively seals around one, two, three or more elongated elements inserted therethrough.

Still another significant aspect and feature of the present invention is a compression sealing mechanism which operates to compress a resilient seal into sealing relationship with elongated elements passing therethrough and by which the degree of compression of the resilient seal can be varied.

Yet another significant aspect and feature of the present invention is a compression sealing mechanism which includes an imperforate seal that seals around elements pushed therethrough.

Having thus described embodiments of the present invention and enumerated significant aspects and features thereof, it is the principal object of the present invention to provide a gas inflation/evacuation system incorporating a reservoir and a removably attached sealing system incorporating a compression sealing mechanism and an inflation tube sealing/crimping mechanism for a guidewire assembly having an occlusive device.

### BRIEF DESCRIPTION OF THE DRAWINGS

Other objects of the present invention and many of the attendant advantages of the present invention will be readily appreciated as the same becomes better understood by reference to the following detailed description when considered in connection with the accompanying drawings, in which like reference numerals designate like parts throughout the figures thereof and wherein:

FIG. 1 is an expanded schematic diagram of a gas inflation/evacuation system incorporating a reservoir and removably attached sealing system for a guidewire assembly having an occlusive device in accordance with the present invention;

FIG. 2 is an exploded isometric view of the inflation tube sealing/crimping mechanism and, in alignment, an isometric view of the compression sealing mechanism;

FIG. 3 is an exploded view of the compression sealing mechanism shown in FIG. 2;

FIG. 4 is an exploded cross section view taken along line 4-4 of FIG. 3;

FIG. 5 is a cross section view of the assembled compression sealing mechanism taken along line 5-5 of FIG. 2;

FIG. 6 is a view showing the inflation tube sealing/crimping mechanism in cross section along the line 6-6 of FIG. 2 in cooperative interaction with the compression sealing mechanism, also shown in cross section, and other components;

FIG. 7 is an assembled schematic diagram of the gas inflation/evacuation system incorporating a reservoir and removably attached sealing system for a guidewire assembly having an occlusive device showing the initial use thereof in accordance with the present invention; and,

FIG. 8 is an assembled schematic diagram of the gas inflation/evacuation system incorporating a reservoir and removably attached sealing system for a guidewire assembly having an occlusive device showing the guidewire assembly separated from the sealing system.

### DETAILED DESCRIPTION OF THE PREFERRED EMBODIMENTS

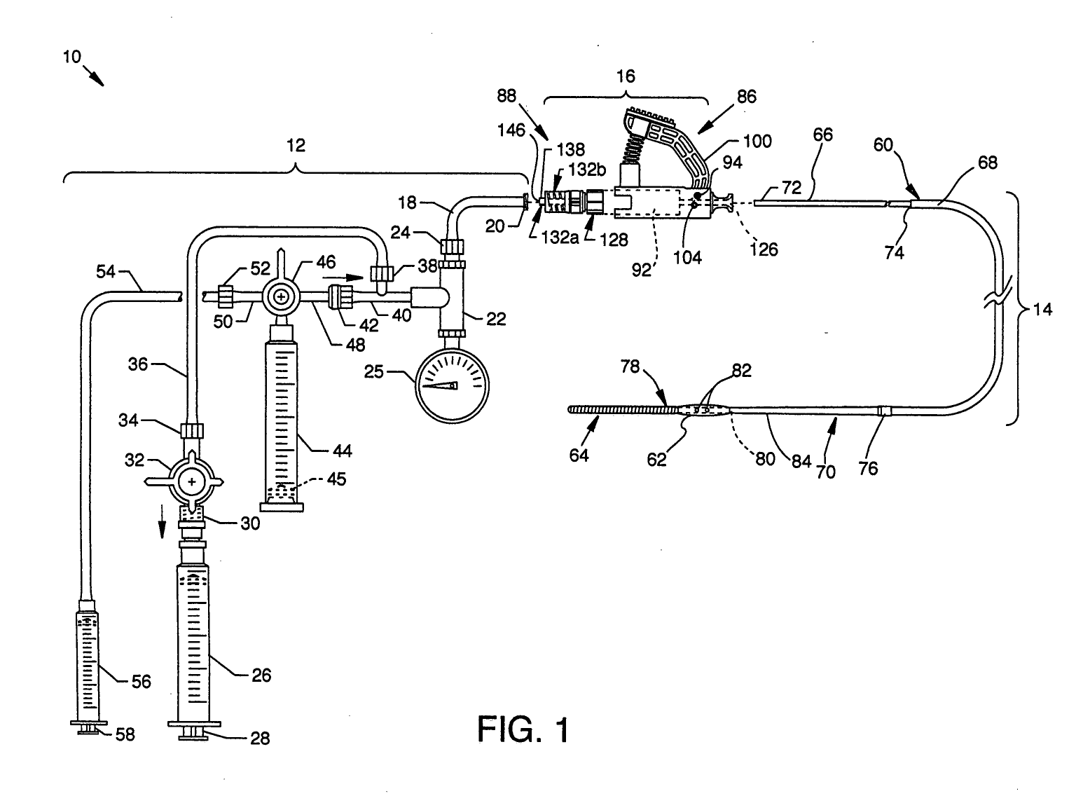

Referring now to FIG. 1, the overall structure and operation of a gas inflation/evacuation system incorporating a reservoir and removably attached sealing system for a guidewire assembly having an occlusive device 10 is now described. The instant invention is comprised of several systems or assemblies including a gas inflation/evacuation system 12, a guidewire assembly 14, and a sealing system 16 which sealingly and removably interfaces and cooperatively interacts between the gas inflation/evacuation system 12 and the guidewire assembly 14. Embodiments of related guidewire occlusion systems are relatingly described in further detail in the previously identified co-pending applications: U.S. patent application Ser. No. 10/838,464, filed Apr. 29, 2004, entitled “Gas Inflation/Evacuation System and Sealing System for Guidewire Assembly Having Occlusive Device,” U.S. patent application Ser. No. 10/012,903, filed Nov. 6, 2001, entitled “Guidewire Occlusion System Utilizing Repeatably Inflatable Gas-Filled Occlusive Device,” U.S. patent application Ser. No. 10/012,891, filed Nov. 6, 2001, entitled “Guidewire Assembly Having Occlusive Device and Repeatably Crimpable Proximal End,” and U.S. patent application Ser. No. 10/007,788, filed Nov. 6, 2001, entitled “Gas Inflation/Evacuation System and Sealing System for Guidewire Assembly Having Occlusive Device.”

The gas inflation/evacuation system 12 preferably includes a valve arrangement that selectively functions as an evacuation system which includes means for evacuating the guidewire assembly 14 and functions as an inflation system which includes means for introducing a biocompatable gas into the guidewire assembly 14 using a flexible conduit 18 having a connector 20 at one end. Another end of the conduit 18 connects to a tee manifold 22 using a connector 24. A pressure gauge 25 is connected to one end of the tee manifold 22. The evacuation system portion of the gas inflation/evacuation system 12 includes an evacuation syringe 26 having a plunger 28, a check valve 30 connected to the evacuation syringe 26 to prevent inadvertent introduction of air into conduits and other components, a positionable evacuation valve 32 connected to the check valve 30, a connector 34 and flexible conduit 36 connected to the positionable evacuation valve 32, a connector 38 connecting the conduit 36 to a conduit tee 40, the conduit tee 40 having a check valve 42 at one end and being connected at the other end to the tee manifold 22, the connector 24, the conduit 18, and the connector 20 which are connectingly utilized to evacuate the guidewire assembly 14. The conduit tee 40, the tee manifold 22, the connector 24, the conduit 18, and the connector 20 are common to the gas inflation system portion of the gas inflation/evacuation system 12. The gas inflation system portion of the gas inflation/evacuation system 12 includes a reservoir 44 having a displaceable piston 45, the reservoir containing a volume of a biocompatable gas sufficient to inflate an occlusive balloon a plurality of times. Preferably, the biocompatable gas is carbon dioxide. Other biocompatable gases that may be utilized with the present invention include helium and nitrous oxide. The reservoir 44 connects to other parts of the gas inflation system portion of the gas inflation/evacuation system 12 including a positionable inflation valve 46 which is attached at one side to a flexible conduit 48 connecting the positionable inflation valve 46 to one end of the check valve 42 and which is attached at the other side to a conduit 50 connecting the positionable inflation valve 46 to one end of a flexible conduit 54 by a connector 52. The other end of the flexible conduit 54 is suitably attached to an inflation syringe 56 having a plunger 58. The gas inflation/evacuation system 12 is connected via conduit 18 to the compression sealing mechanism 88 of the sealing system 16, as later described in detail.

The guidewire assembly 14 includes a guidewire 60, an occlusive device such as an occlusive balloon 62, and, optionally, a flexible tip 64. The guidewire 60 is tubular and comprises an extended sealable section 66, a main body portion 68, a distal portion 70, and a proximal end 72. Preferably, the extended sealable section 66 can be a drawn down portion of the main body portion 68 or can be a separate piece which extends from the proximal end 72 of the guidewire 60 to the main body portion 68 to which it is joined, preferably by a laser weld 74. The distal portion 70 which preferably is Ni—Ti is generally a separate piece which is joined to the main body portion 68, preferably by a stainless steel sleeve 76, and extends distally from the main body portion 68 to the distal end 78 of the guidewire 60. As used in the present invention, the terms proximal and distal will be used with reference to an operator, such that a distal portion of the guidewire 60, for example, is the portion first inserted into a blood vessel, and the proximal portion remains exterior to the patient and is therefore closer to the operator. Preferably, the extended sealable section 66 is an extended crimpable section comprised of a tubular segment having an outer diameter smaller than an outer diameter of the main body portion 68 of guidewire 60. Although the diameter of the extended crimpable section could be any size consistent with effective use as a guidewire, it will be understood that the smaller diameter allows for less force to be used in sealing the extended crimpable section and provides a crimped seal that is not too large when crimped. The occlusive balloon 62 is located along the distal portion 70 of guidewire 60. The occlusive balloon 62 is fluidly connected via a lumen 80 to the proximal end 72 of the guidewire 60, with channels or holes 82 allowing for fluid communication between lumen 80 and occlusive balloon 62. In a preferred embodiment, the flexible tip 64 is used and is located at the distal end of the guidewire assembly 14. Preferably, the distal portion 70 of guidewire 60 includes a tapered portion 84 to increase the flexibility and transition properties of the distal portion 70.

In a preferred embodiment, guidewire assembly 14 is constructed as described in further detail in the previously identified co-pending application, U.S. patent application Ser. No. 10/012,891 entitled “Guidewire Assembly Having Occlusive Device And Repeatably Crimpable Proximal End.” For example, and for the purpose of demonstration, the main body portion 68 is formed of a stainless steel hypotube having an outer diameter of 0.013 inch and an inner diameter of 0.007 inch. To accomplish passive deflation in the desired time of less than one minute when the extended sealable section 66 is cut, it is preferable that the main body portion 68 have an inner diameter of at least 0.004 inch. The extended sealable section 66 of guidewire 60 is comprised of a crimp tube which is also formed of stainless steel and having an outer diameter of 0.009 inch to 0.012 inch and an inner diameter of at least 0.003 inch and preferably about 0.005 inch. As mentioned before, the extended sealable section 66 is generally a separate piece secured to the main body portion 68 by a laser weld 74 or may be formed by centerless grinding or reducing the outer diameter of a portion of the proximal portion of the main body portion 68 of the guidewire 60. Still other embodiments may enable the extended sealable section to be a modified, treated or otherwise fabricated portion of the proximal portion of the main body portion 68 that is suitable for the particular sealing technique to be used.

The extended sealable section 66 can be made of any material that when deformed and severed retains that deformation so as to form an airtight seal. When crimped and severed, it is preferable that the extended sealable section 66 not present a sharp, rigid point that is capable of piercing a gloved hand. It has been found that so long as the preferred embodiment is not gripped within less than one inch of the proximal end of the extended sealable section 66, the severed proximal end of the extended sealable section 66 does not penetrate a standard surgical glove. In addition, the extended sealable section 66 must have sufficient strength in terms of high tensile and kink resistance to permit catheter devices to repeatedly pass over the extended sealable section 66.

The main body portion 68 is preferably secured to the distal portion 70 using a Ni—Ti or stainless steel sleeve 76 laser welded to the main body portion 68 and crimped to the distal portion 70. The distal portion 70 is preferably formed of a Ni—Ti alloy having an inner diameter of 0.0045 inch and an outer diameter that ranges from 0.014 inch to 0.0075 inch to form tapered portion 84, preferably formed by a centerless grinding process. The flexible tip 64 is a coiled tip attached to distal portion 70 distal to occlusive balloon 62, preferably by crimping. Alternatively, a sleeve could be welded to the flexible tip 64, and the tapered portion 84 could then be inserted into this sleeve and secured thereto by crimping or by the use of adhesive.

Alternatively, any number of other alloys or polymer materials and attachment techniques could be used in the construction of the guidewire 60, provided the materials offer the flexibility and torque characteristics required for a guidewire and the attachment techniques are sufficiently strong enough and capable of making an airtight seal. These materials include, but are not limited to, Ni—Ti, 17-7 stainless steel, 304 stainless steel, cobalt superalloys, or other polymer, braided or alloy materials. The attachment techniques for constructing guidewire 60 include, but are not limited to, welding, mechanical fits, adhesives, sleeve arrangements, or any combination thereof.

The occlusive balloon 62 may be made of any number of polymer or rubber materials. Preferably, the occlusive balloon is preinflated to prestretch it so that expansion is more linear with pressure. Preferably, the pressure supplied by gas inflation/evacuation system 12 is designed to stay well within the elastic limit of the occlusive balloon 62. A two-layer occlusive balloon arrangement, adding gas and/or liquid between balloon layers, may be used in an alternate embodiment to increase visibility of the distal end 78 of the guidewire 60 under fluoroscopy.

The sealing system 16, further illustrated later in detail, includes an inflation tube sealing/crimping mechanism 86 and a compression sealing mechanism 88 which cooperatively interact with each other and which temporarily connect between and cooperatively interact with the gas inflation/evacuation system 12 and the guidewire assembly 14 during a vascular medical procedure for removal of thrombus and the like.

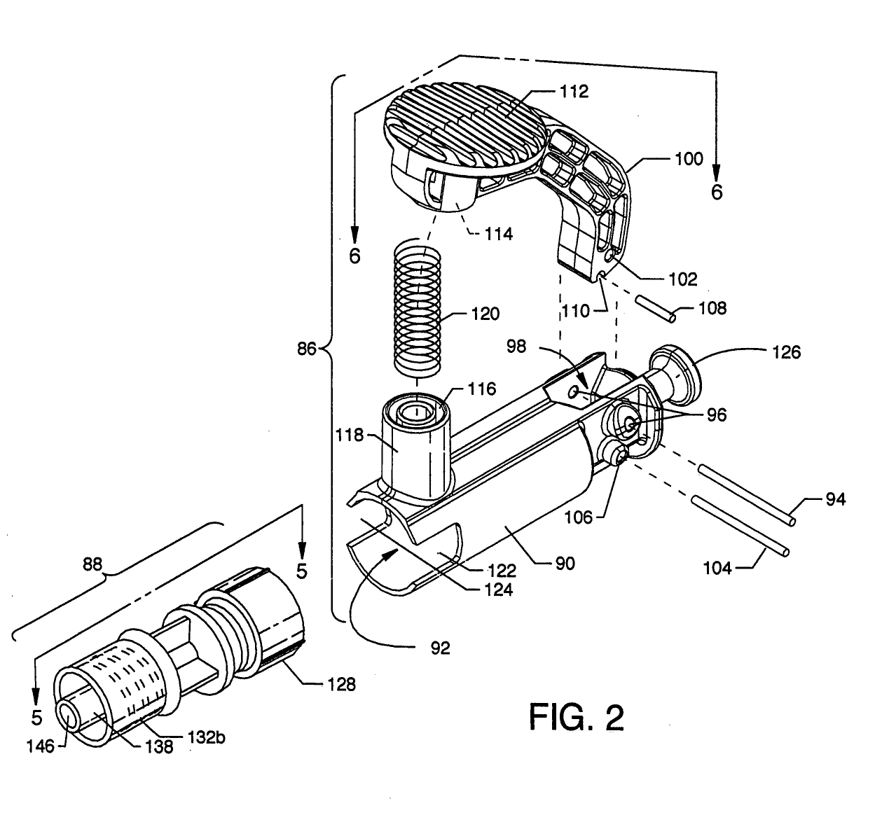

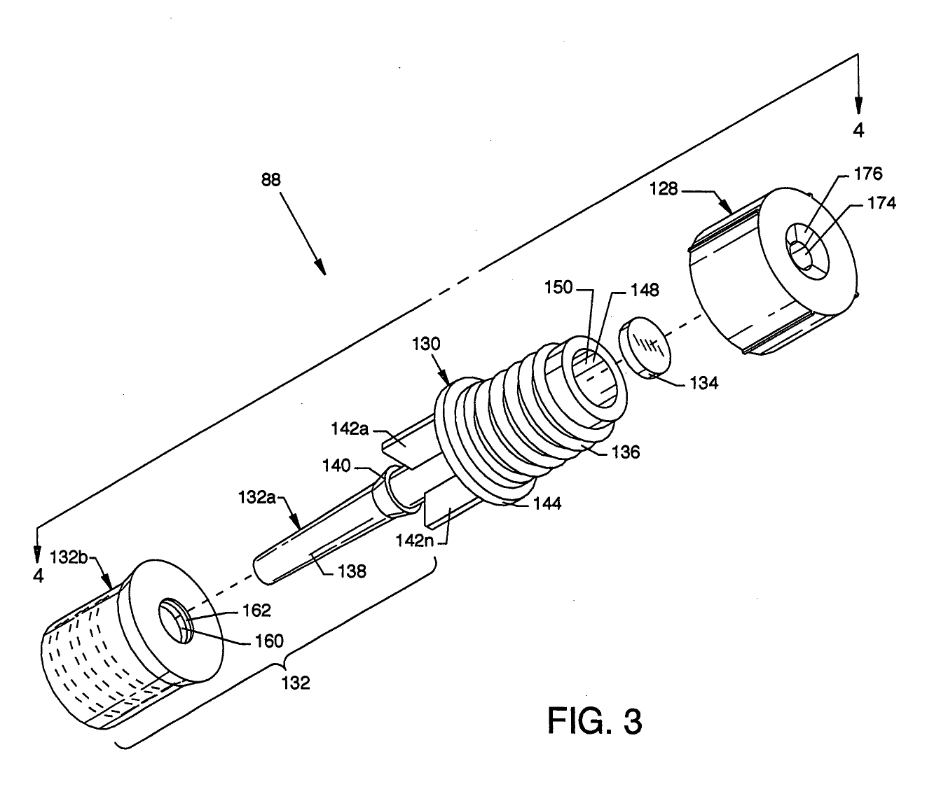

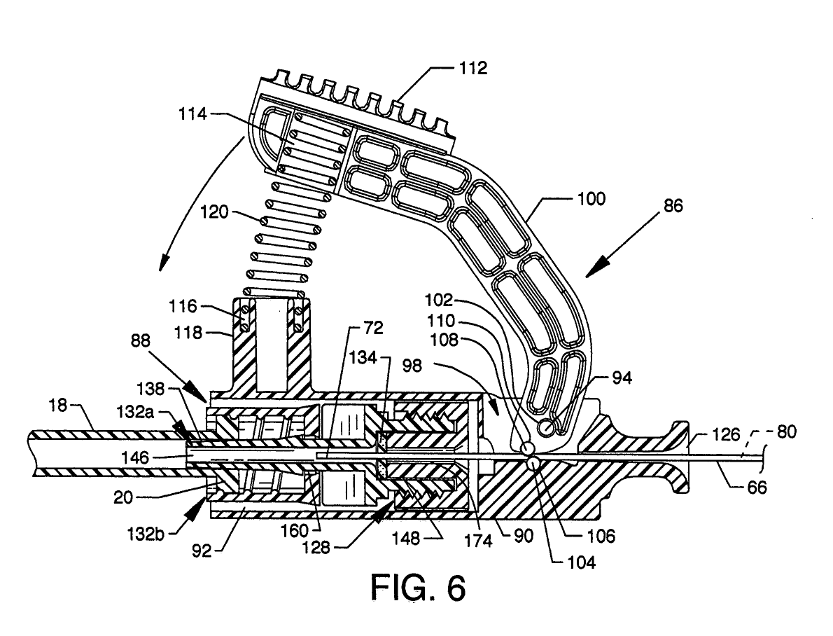

FIG. 2 is an exploded isometric view of the inflation tube sealing/crimping mechanism 86 and, in alignment, an isometric view of the compression sealing mechanism 88. The inflation tube sealing/crimping mechanism 86 includes a configured body 90 being generally tubular in shape and including a centrally located passageway 92 for mated accommodation of the sealing cap 128 of the compression sealing mechanism 88 therein. Also included is a pivot dowel pin 94, preferably of hardened steel, which aligns through a hole set 96 and through a cavity 98 which extends along and across one end of the body 90 for accommodation of the lower end of a geometrically configured pivotal handle 100, as well as extending through a horizontally oriented pivot hole 102 located in the lower region of the pivotal handle 100. A stationary pincer dowel pin 104, preferably of hardened steel, aligns in a transversely oriented hole 106, the central part of which is truncated, where the truncated hole portion is located at the bottom of the cavity 98. The upper region of the stationary pincer dowel pin 104 protrudes slightly above the central truncated portion of the hole 106 and above the lower surface of the cavity 98, as shown in FIG. 6, in order to accommodate a surface of the extended sealable section 66 of the guidewire assembly 14. An actuated pincer dowel pin 108, preferably of hardened steel, aligns and affixes to a truncated hole 110 at the lower region of the pivotal handle 100 and protrudes slightly below the truncated portion of the truncated hole 110 and below the lower surface of the lower region of the pivotal handle 100. An actuating pad 112, preferably having a tactile surface, is located at the upper end of the pivotal handle 100 in close proximity to a spring receptor cavity 114. Another spring receptor cavity 116, being annular in shape, is located in a cylindrical post 118 extending in vertical orientation from the end of the body 90. Opposing ends of a return spring 120 mount in and between the spring receptor cavity 114 and the spring receptor cavity 116 to forcibly position the pivotal handle 100 to an open position with respect to the actuated pincer dowel pin 108 and the stationary pincer dowel pin 104 for accommodation of the extended sealable section 66 of the guidewire assembly 14. Horizontally opposed notches 122 and 124 are located in one end of the body 90 to accommodate other structure, if required. An elongated receptor orifice 126 is located at the distal end of the configured body 90 in aligned communication with the passageway 92 for accommodation of one end of the extended sealable section 66 of the guidewire assembly 14. The compression sealing mechanism 88, which is fittingly accommodated by the passageway 92, is described with reference to FIGS. 3, 4 and 5.

FIG. 3 is an exploded view of the compression sealing mechanism 88 shown in FIG. 2. FIG. 3, in addition to showing the sealing cap 128, shows a one-piece sealing gland 130, a fluid connector 132, and a seal 134 constructed of a resilient material, preferably silicone. The seal 134 is imperforate, axially compressible, and sealingly radially expandable, and is shaped as a disc having two substantially flat faces substantially parallel to each other and separated by a thickness. The sealing gland 130 serves as a mount for the sealing cap 128 and together with the sealing cap 128 constitutes a compressing apparatus for compressing the seal 134. Sealing gland 130 also serves as a mount for the fluid connector 132. Fluid connector 132 is illustrated as a male Luer connector with a Luer taper component 132a, which forms a fluid seal with the conduit 18 (FIG. 6), and a threaded locking component 132b, which provides a secure mechanical attachment to the conduit 18. One end of the sealing gland 130 includes external threads 136 for receiving and mounting the sealing cap 128. Located along the longitudinal axis of the sealing gland 130 at the end thereof opposite to the external threads 136 is a connector tube 138 which forms a portion of the Luer taper component 132a and which has an annular ramp 140 over which the threaded locking component 132b is mounted by snap engagement and by which the threaded locking component 132b is captured and rotatably retained upon the connector tube 138 distal to the annular ramp 140 and adjacent to a plurality of support struts 142a-142n extending along a portion of the connector tube 138 and terminating at an annular ridge 144, which is shown to be continuous, but which could be spaced segments, for manual grasping.

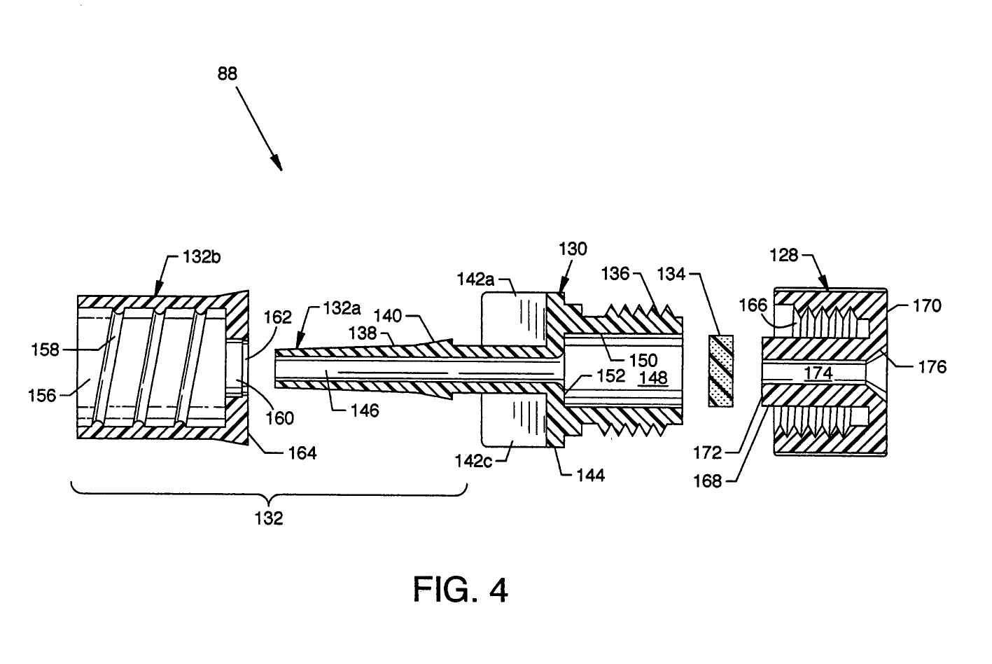

FIG. 4 is an exploded cross section view taken along line 4-4 of FIG. 3. Shown in particular is a distally located cavity 148 extending along and about the longitudinal axis of the sealing gland 130. The cavity 148 includes a circular peripheral wall 150 intersecting a surface in the form of a sealing seat 152 which is planar in nature. A passageway 146 extends partially along and about the longitudinal axis of the sealing gland 130 and within the connector tube 138 in communication with the cavity 148. The threaded locking component 132b of the fluid connector 132 includes an interior cavity 156 which is tubular and includes a raised threaded surface 158 for accommodation and fixation to desired appliances. A hole 160 having an annular ridge 162 is located in the end wall 164 of the threaded locking component 132b. The annular ridge 162 snappingly engages over and about the annular ramp 140 of the connector tube 138 to rotatably retain the threaded locking component 132b upon the connector tube 138, as previously described. The sealing cap 128 includes internal threads 166 suitable for threadingly engaging the external threads 136 of the sealing gland 130. A tubular extension 168 extends proximally from the end wall 170 of the sealing cap 128 and terminates in an annular planar sealing face 172. A passageway 174 having a distally located annular bevel 176 extends from the end wall 170 through the tubular extension 168 and intersects the annular planar sealing face 172.

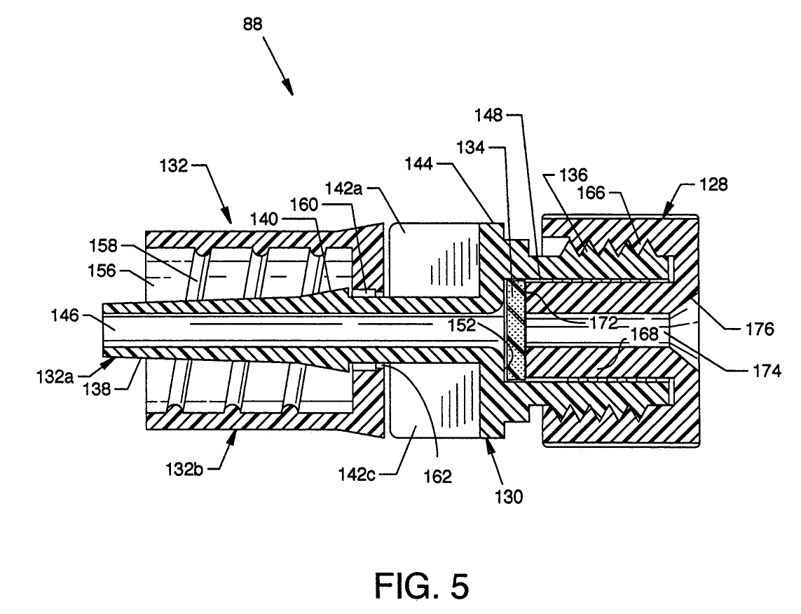

FIG. 5 is a cross section view of the assembled compression sealing mechanism 88 taken along line 5-5 of FIG. 2, and FIG. 6 is a view showing the inflation tube sealing/crimping mechanism 86 in cross section along the line 6-6 of FIG. 2 in cooperative interaction with the compression sealing mechanism 88, also shown in cross section, and other components. In use, the seal 134 is aligned in the cavity 148 preferably in initial contact with the sealing seat 152 followed by threaded engagement of the sealing cap 128 to the sealing gland 130 to capture the seal 134. The extended sealable section 66 of the guidewire 60 can then be introduced and guided by the annular bevel 176 into the passageway 174 of the sealing cap 128, and thence pushed through the seal 134 and into the passageway 146 of the connector tube 138. The extended sealable section 66 of the guidewire 60 maintains a coaxial relationship to the annular bevel 176, the passageway 174, the seal 134, the cavity 148, the passageway 146, the annular ridge 162, the hole 160, and the interior cavity 156 of the threaded locking component 132b, all of which have a mutual coaxial relationship along a central longitudinal axis. The internal threads 166 of the sealing cap 128 threadingly engage the external threads 136 of the sealing gland 130, and the sealing cap 128 is rotatably advanced with respect to the sealing gland 130 to bring the annular sealing face 172 of the tubular extension 168 into intimate contact with the seal 134. Such advancing rotation causes the sealing face 172 of the sealing cap 128 to forcibly engage the seal 134 to compress the seal 134 between the sealing face 172 of the sealing cap 128 and the sealing seat 152 of the sealing gland 130 to cause inwardly directed closure movement of the seal 134 and to expand the periphery of the seal 134 radially and outwardly, thereby providing a seal against the extended sealable section 66 of the guidewire and against the circular peripheral wall 150 of the sealing gland 130, respectively. The compression sealing mechanism 88 reversibly lockably fits into the inflation tube sealing/crimping mechanism 86. The fluid connector 132 of the compression sealing mechanism 88 receives the connector 20 of the conduit 18, which is connectible to the gas inflation/evacuation system 12. The guidewire assembly extended sealable section 66 is passed through the elongated receptor orifice 126 to pass through the inflation tube sealing/crimping mechanism 86, to pass through the seal 134, and to extend into the passageway 146 of the connector tube 138. The seal 134 seals around and about the extended sealable section 66 to await evacuation of the guidewire assembly 14 and various regions of the gas inflation/evacuation system 12 and subsequent inflation of the occlusive balloon 62 followed by crimping of the extended sealable section 66. During crimping, the extended sealable section 66 is sealingly severed by action of the pivotal handle 100 to maintain the inflation of the occlusive balloon 62, whereby the guidewire assembly 14 can be utilized in a medical interventional procedure after which the sealed and crimped end of the extended sealable section 66 may be severed to accomplish deflation of the occlusive balloon 62. Subsequently, a remaining portion of the extended sealable section 66 may be reinserted through the seal 134 for additional inflation, deflation and sealing sequences. The breaking off and reinsertion process may be repeated as necessary.

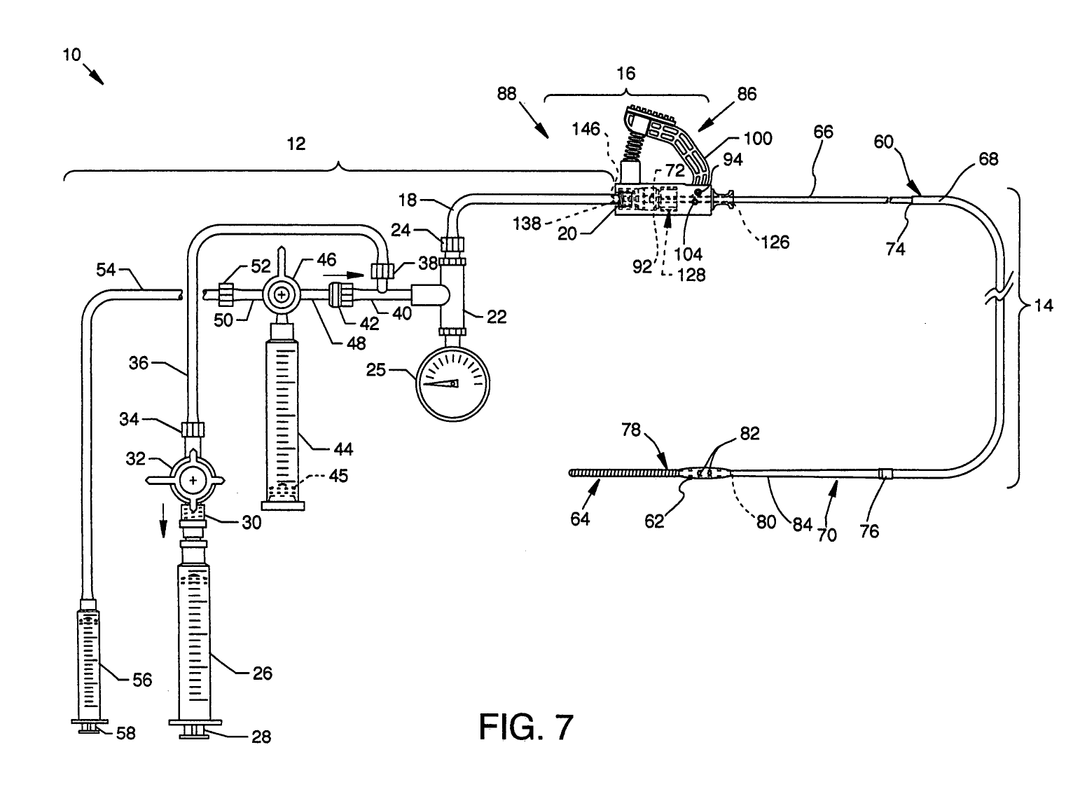

FIG. 7 is an assembled schematic diagram of the gas inflation/evacuation system incorporating a reservoir and removably attached sealing system for a guidewire assembly having an occlusive device 10 showing the initial use thereof in accordance with the present invention.

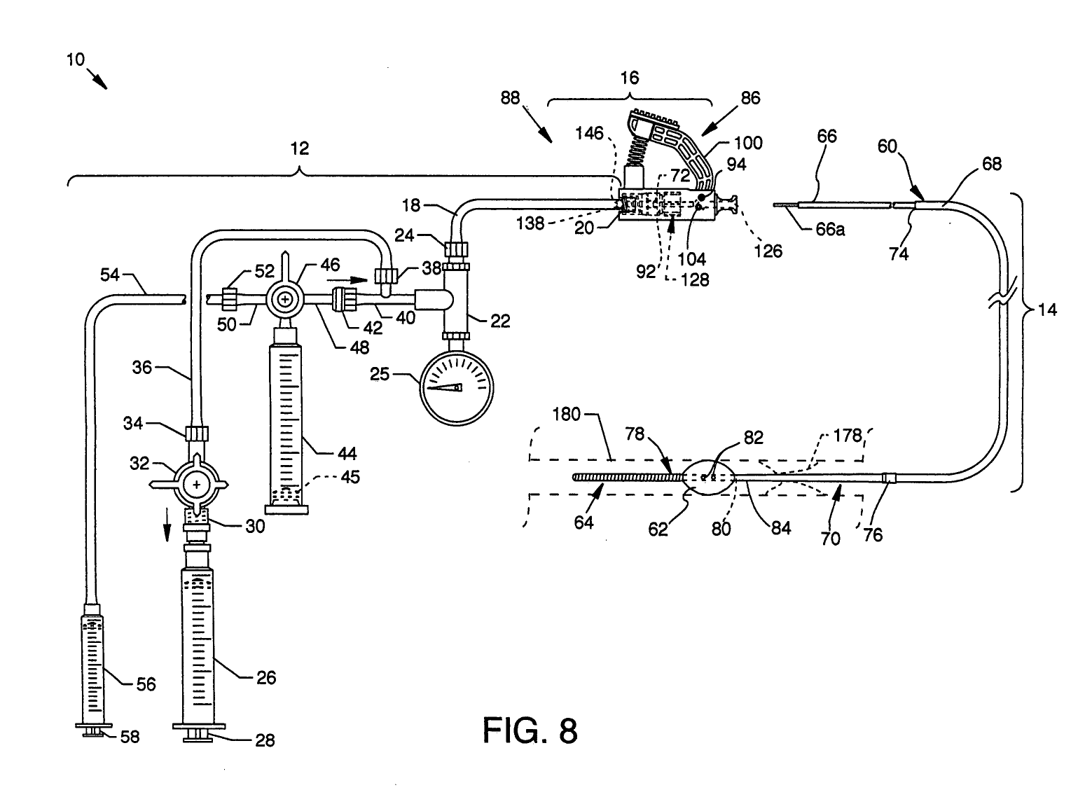

FIG. 8 is an assembled schematic diagram of the gas inflation/evacuation system incorporating a reservoir and removably attached sealing system for a guidewire assembly having an occlusive device 10 showing the guidewire assembly 14 separated from the sealing system 16.

### Mode of Operation

The instant invention is generally used in the following manner where a patient is prepared for a common interventional procedure involving the ablative removal of thrombus, plaque, lesions and the like, or placing a stent, or common angioplasty, or any procedure that may produce particles that will be removed later, such as by cross stream or other thrombectomy catheter device, for instance, via a femoral arterial access or other suitable site. The guidewire assembly 14 is inserted alone or through a prepositioned sheath, a guide catheter, or introducer and is tracked to a preferred location distal to the buildup site. Subsequent to such positioning, the occlusive balloon 62 is repeatedly inflated and deflated as required to controllingly and appropriately allow blood flow, to actively function as an occlusive device, and to serve as a guidewire for loading of and use with ablation catheter devices, for placement of stents, or for other procedures. Subsequent to guidewire placement, the proximal end 72 of the guidewire 60 is loaded into inflation tube sealing/crimping mechanism 86 and into the compression sealing mechanism 88. Then, vacuum is utilized to purge the system of air, the inflation tube sealing/crimping mechanism 86 then allows for metered biocompatable, highly blood soluble gas such as CO₂, helium, or other biocompatable gas to be introduced to inflate the occlusive balloon 62 to a desired size, the inflation tube sealing/crimping mechanism 86 is activated to seal and sever the extended sealable section 66 of the guidewire 60, the guidewire 60 then is removed from the inflation tube sealing/crimping mechanism 86, and the guidewire 60 is then used with any other compatible interventional device, such as a thrombectomy catheter, a stent, or other device in the manner desired. Thus, having a basic understanding of the present invention, the following steps in the mode and method of operation are now described with particular reference to FIG. 6, FIG. 7 and FIG. 8 and with understood reference to the other illustrations:

1. The proximal end 72, more specifically the extended sealable section 66, is inserted into the elongated receptor orifice 126 of the inflation tube sealing/crimping mechanism 86 until passing through the seal 134 of the compression sealing mechanism 88 (FIG. 6).

2. The positionable inflation valve 46 is positioned to disrupt communication between the conduit 48 and the reservoir 44 and then the positionable evacuation valve 32 is positioned to allow communication between the evacuation syringe 26, the check valve 30, the positionable evacuation valve 32, the conduit 36, the conduit tee 40 and thus to one side of the check valve 42 and to the tee manifold 22, the pressure gauge 25, the connector 24, the conduit 18, the passageway 146 of the compression sealing mechanism 88 (FIG. 6), and the guidewire assembly 14 including the extended sealable section 66 of the guidewire 60, the lumen 80 of the guidewire 60, the holes 82 of the guidewire 60 and the occlusive balloon 62.

3. The plunger 28 of the evacuation syringe 26 is withdrawn to evacuate the guidewire assembly 14 and other communicating members where the vacuum (or pressure) is observed on the pressure gauge 25.

4. The positionable evacuation valve 32 is then closed wherein the guidewire assembly 14 maintains an evacuated state by action of the closed positionable evacuation valve 32.

5. The positionable inflation valve 46 is actuated to allow communication of the biocompatable inflation medium in the reservoir 44 with the positionable inflation valve 46, the conduit 50, the connector 52, the conduit 54, and the inflation syringe 56.

6. The plunger 58 is withdrawn to load the biocompatable inflation medium from the reservoir 44 into the inflation syringe 56 where appropriate used volumes can be observed by viewing the displaceable piston 45 located in the reservoir 44 and by observing the inflation syringe 56.

7. The positionable inflation valve 46 is positioned to disallow communication from the reservoir 44 and to allow pressurized communication from the biocompatable inflation-medium-laden inflation syringe 56 with and through the conduit 54, the conduit 50, the positionable inflation valve 46, the conduit 48, the check valve 42, the conduit tee 40, the tee manifold 22, the conduit 18, the passageway 146 of the compression sealing mechanism 88 (FIG. 6), and with and through the guidewire assembly 14 including the extended sealable section 66 of the guidewire 60, the lumen 80 of the guidewire 60, the holes 82, and the occlusive balloon 62.

8. The plunger 58 of the inflation syringe 56 is depressed to dispel and urge biocompatable inflation and along the route just described while observing the pressure gauge 25.

9. Upon desired inflation of the occlusive balloon 62 the pivotal handle 100 of the inflation tube sealing/crimping mechanism 86 is actuated to crimp and sever the extended sealable section 66 of the guidewire 60 to form a crimped sealed section 66a whereby the crimped sealed section 66a maintains the pressure within the lumen 80 for maintained inflation of the occlusive balloon 62.

10. The crimped sealed section 66a is removed from influence of the inflation tube sealing/crimping mechanism 86 and the compression sealing mechanism 88 to reveal the crimped sealed section 66a as shown in FIG. 8 where the occlusive balloon 62 is shown in the inflated position and downstream from a thrombus deposit 178 within a vascular member 180, each representatively shown by dashed lines in FIG. 8. The guidewire assembly 14 is now ready for guidance of other devices such as catheters, thrombectomy catheters, stents and the like to a vascular site proximal of the occlusive balloon 62.

11. Subsequent to usage of the guidewire assembly 14, the occlusive balloon 62 is deflated by severing the guidewire 60, such as by the use of a scissors, just distal to the crimped sealed section 66a. An increased deflation rate as well as a further reduction of the physical cross section of the occlusive balloon 62 can be accomplished by reinserting the newly severed and newly defined proximal end 72 of the guidewire 60 (the extended sealable section 66) into the inflation tube sealing/crimping mechanism 86 and compression sealing mechanism 88 and accomplishing the steps outlined in steps 1, 2 and 3 above.

12. Further and repeated use of the invention is accomplished by repetition of steps 1 through 11 until the content of the reservoir 44 is depleted or until the extended sealable section 66 is of insufficient length for use, whichever occurs first.

Various modifications can be made to the present invention without departing from the apparent scope thereof.

It is claimed:

1. A gas inflation/evacuation system for a catheter guidewire assembly defining a lumen and having a proximal portion and a distal portion having an occlusive device, the gas inflation/evacuation system comprising:
a. a reservoir; and,
b. a removably attached sealing system for interfacing and cooperatively interacting with the proximal portion and lumen of the catheter guidewire assembly having the occlusive device.

2. The gas inflation/evacuation system of claim 1, wherein the reservoir includes a biocompatable gas.

3. The gas inflation/evacuation system of claim 2, wherein the biocompatable gas is selected from the group consisting of carbon dioxide, helium, and nitrous oxide.

4. A gas inflation/evacuation system for a guidewire assembly having a proximal portion and a distal end and defining a guidewire lumen with an occlusive balloon located proximate the distal end of the guidewire assembly and communicating with the guidewire lumen, the gas inflation/evacuation system comprising:
a. a first syringe arrangement having an evacuation syringe for selectively evacuating the guidewire lumen;
b. a reservoir arrangement for supply of an abundance of biocompatable gas inflation medium from a reservoir for multiple inflations of the occlusive balloon;
c. a second syringe arrangement including an inflation syringe which selectively communicates with the reservoir for biocompatable gas inflation medium supply and which selectively introduces biocompatable gas inflation medium into the guidewire lumen to inflate the occlusive balloon in fluid communication with the guidewire lumen of the guidewire assembly; and,
d. a compression sealing mechanism removably connected to the proximal portion of the guidewire assembly to provide a sealed interface between the guidewire lumen of the guidewire assembly and the first and second syringe assemblies where such sealed interface is suitably sealed to allow inflation of the occlusive balloon.

5. The gas inflation/evacuation system of claim 4, wherein the proximal portion of the guidewire assembly includes a proximally located extended sealable section, and wherein the compression sealing mechanism can seal around multiple elongated elements that puncture a seal residing in the compression sealing mechanism so that the compression sealing mechanism can be used a plurality of times without replacing internal seals of the compression sealing mechanism.

6. The gas inflation/evacuation system of claim 5, wherein the compression sealing mechanism may be used to seal around the extended sealable section of the guidewire of the guidewire assembly that cooperates with the gas inflation/evacuation system located proximal to the seal located internally within the compression sealing mechanism.

7. The gas inflation/evacuation system of claim 6, wherein the sealing system also includes an inflation tube sealing/crimping mechanism which accommodatingly receives the proximal portion of the guidewire assembly including the extended sealable section for crimping and sealing during use of the invention.

8. The gas inflation/evacuation system of claim 7, wherein the inflation tube sealing/crimping mechanism also can sever the crimped sealed section of the guidewire subsequent to desired inflation of the occlusive balloon at the distal location on the guidewire to maintain the occlusive balloon in an inflated state and to present a proximal guidewire end unencumbered by external interfering structure suitable for accommodation of a catheter, a hub or other such devices requiring the use of guidance to a vascular site requiring the removal of thrombus, lesion, or plaque.

9. The gas inflation/evacuation system of claim 8, wherein the portion of the crimped sealed section immediately distal of the crimped sealed portion can be cut to open communication to the lumen, thereby allowing the occlusive balloon to readily deflated.

10. The gas inflation/evacuation system of claim 9, wherein the remaining intact proximal portion of the guidewire can subsequently be reinserted into the seal to re-engage the gas inflation/evacuation system for another inflation of the occlusive balloon, whereby the seal effectively seals around the remaining extended sealable section of the guidewire assembly.

11. The gas inflation/evacuation system of claim 4, wherein the compression sealing mechanism can be removably connected to the proximal portion of the guidewire assembly between two and ten times.

12. The gas inflation/evacuation system of claim 4, wherein the compression sealing mechanism can be removably connected to the proximal portion of the guidewire assembly at least three times.

13. The gas inflation/evacuation system of claim 10, wherein the seal is compressed by an assembly of axially related backing or retaining members that include sealing surfaces that contact the seal and apply a compressive force to the seal.

14. The gas inflation/evacuation system of claim 13, wherein the compressive force is generated by a threaded member such as nuts, caps, screw followers, and sealing glands or is generated by springs, tensioned parts, and biased members.

15. The gas inflation/evacuation system of claim 13, wherein the seal is formed of a resilient material.

16. The gas inflation/evacuation system of claim 13, wherein the seal has a thickness of from about 0.030 inch to about 0.200 inch.

17. The gas inflation/evacuation system of claim 4, wherein the inflation syringe is appropriately sized to contain just enough inflation medium to inflate the occlusive balloon so as to minimize the volume of biocompatable gas in the gas inflation/evacuation system in the event of a leak.

18. The gas inflation/evacuation system of claim 4, further comprising:
a. at least one check valve to prevent undesired communication direction;
b. a pressure gauge to monitor pressure; and,
c. at least one positionable valve.

19. A method of controlling an occlusive balloon on a guidewire assembly comprising the steps of:
a. providing a gas inflation/evacuation system for a guidewire assembly having a proximal portion and a distal end and defining a guidewire lumen with an occlusive balloon located proximate the distal end of the guidewire assembly and communicating with the guidewire lumen, the gas inflation/evacuation system including:
(1) a first syringe arrangement having an evacuation syringe for selectively evacuating the guidewire lumen;
(2) a reservoir arrangement for supply of an abundance of biocompatable gas inflation medium from a reservoir for multiple inflations of the occlusive balloon;
(3) a second syringe arrangement including an inflation syringe which selectively communicates with the reservoir for biocompatable gas inflation medium supply and which selectively introduces biocompatable gas inflation medium into the guidewire lumen to inflate the occlusive balloon in fluid communication with the guidewire lumen of the guidewire assembly; and,
(4) a compression sealing mechanism removably connected to the proximal portion of the guidewire assembly to provide a sealed interface between the guidewire lumen of the guidewire assembly and the first and second syringe assemblies where such sealed interface is suitably sealed to allow inflation of the occlusive balloon;
b. inflating the occlusive balloon with the second syringe arrangement and crimp sealing the proximal portion of the guidewire assembly and separating the gas inflation/evacuation system from the guidewire assembly;
c. severing the crimp sealed proximal portion and evacuating the occlusive balloon; and,
d. re-inflating the occlusive balloon with the second syringe arrangement and re-crimp sealing the proximal portion of the guidewire assembly.

20. The method of claim 19, wherein the step of evacuating the occlusive balloon is preceded by a step of:
a. reinserting the severed proximal portion into the compression sealing mechanism of the inflation/evacuation system; and,
wherein the step of evacuation is characterized by a deflation rate of the occlusive balloon being increased by employing the first syringe arrangement.
